(19) United States
(12) Patent Application Publication
(10) Pub. No.: US 2019/0201316 A1
(43) Pub. Date: Jul. 4, 2019

(54) HAIR TREATMENT AGENTS

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 16/284,277

(22) Filed: Feb. 25, 2019

Related U.S. Application Data
(63) Continuation of application No. 15/366,344, filed on Dec. 1, 2016.

(30) Foreign Application Priority Data
Dec. 1, 2015 (DE) 10 2015 223 818.0

(51) Int. Cl. A61K 8/898; A61K 8/41; A61K 8/36; A61Q 5/00; A61Q 5/12; A61Q 5/02; A61K 8/34

(52) U.S. Cl. CPC A61K 8/898; A61K 8/416; A61K 8/361; A61K 8/342; A61Q 5/12; A61Q 5/02; A61Q 5/004

(57) ABSTRACT

Hair treatment agents including: at least one fatty alcohol; at least one cationic surfactant; at least one silicone; and 10-undecenoic acid and/or salts thereof.

### BACKGROUND

The present invention relates to hair treatment agents. In particular, to shampoos and conditioners having active ingredients for hair care.

The importance of hair care products with the longer performance has grown. This is due in part to increased stress on hair, for example, from dyeing, permanent waves, cleaning of hair with shampoos, and due to environmental pollution. Such hair care products have an influence on the natural structure and properties of hair. For example, the wet and dry combability of hair, the hold and body of hair, and/or protection from increased split ends may be impacted by use of appropriate hair care products.

It has been customary to subject hair to special after-treatments in which the hair is treated with special active ingredients, for example, quaternary ammonium salts or special polymers. This is usually in the form of a rinse. These treatments may result in improved combability, hold, and body of hair while reducing the amount of split ends, depending on the formulation.

Multifunctional cosmetic products are also known in the prior art. In particular, this includes “two-in-one” shampoos, which clean and condition the hair. Such products are appreciated by consumers because the product eliminates the need for at least one procedural step, e.g., conditioning with a traditional hair conditioner.

### DETAILED DESCRIPTION

Products for changing the natural hair color likewise play a prominent role in hair cosmetics. A distinction is made between permanent, semipermanent, and temporary dyeing systems, which are based on chemical and/or natural dyes. However, the hair colors produced artificially by means of permanent, semipermanent, or temporary dyeing systems have the disadvantage that said hair colors can change in an undesired manner, for example during or after hair cleaning.

“Undesired change” is understood to mean fading or bleeding out and the loss of the color brilliance of the shade of color of the hair achieved by means of the particular coloring. Environmental influences and/or sun exposure can increase these changes.

The use of divalent metal salts in hair dyeing agents to improve the durability and thus the fastness of the coloring is known from EP 2438900 A1, which is incorporated by reference herein.

There is still a need to provide active substances or active-substance combinations for hair treatment agents having good care properties that also strengthen the adherence of dyes to the hair fibers and thus maintain the fastness of the artificially produced hair color and to further develop hair treatment agents in this regard.

It has now been found that a combination of certain ingredients has an especially positive effect on dyed hair treated with said combination and on the hair follicle.

First, the present invention relates to hair treatment agents including: at least one fatty alcohol, at least one cationic surfactant, at least one silicone, and 10-undecenoic acid (undecylenic acid) and/or salts of undecylenic acid.

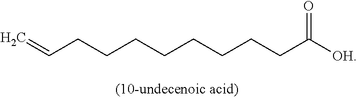

In the sense of the present invention, hair treatment agents include, for example: hair shampoos, hair conditioners, conditioning shampoos, hair sprays, hair conditioning agents, hair masks, hair packs, hair tonics, permanent-wave fixation solutions, hair dyeing shampoos, hair dyeing agents, hair setting lotions, hair styling agents, hair styling preparations, blow-dry curling lotions, mousses, hair gels, hair waxes, and/or combinations thereof. As men in particular often shy away from the use of several different agents and/or several application steps, agents that men use anyway are preferred. Therefore, preferred agents include: shampoos, conditioning media, and/or hair tonics.

The hair treatment agents include at least one fatty alcohol.

Fatty alcohols are aliphatic, long-chain, monohydric primary alcohols having hydrocarbon residues, which have 6 to 30, preferably 6 to 22, carbon atoms. The hydrocarbon residues can be saturated or mono- or polyunsaturated. Fatty alcohols that can be used with preference in the context of the present invention are selected from 1-hexanol, 1-heptanol, 1-octanol, 1-decanol, 1-dodecanol (lauryl alcohol), 1-tetradecanol (myristyl alcohol), 1-hexadecanol (cetyl alcohol), 1-heptadecanol (margaryl alcohol), 1-octadecanol (stearyl alcohol), 1-icosanol (arachidyl alcohol), 1-docosanol (behenyl alcohol), 1-tetracosanol (lignoceryl alcohol), 1-hexacosanol (ceryl alcohol), 1-octacosanol (montanyl alcohol), 1-triacontanol (melissyl alcohol), cis-9-hexadecen-1-ol (palmitoleyl alcohol), cis-9-octadecen-1-ol (oleyl alcohol), trans-9-octadecen-1-ol (elaidyl alcohol), cis-11-octadecen-1-ol, 6,9,12-octadecatrien-1-ol (y-linolenyl alcohol), and mixtures thereof.

Especially preferred hair treatment agents are characterized in that said hair treatment agents include, with respect to the weight of the agent, 0.1 to 20 wt. %, preferably 0.5 to 15 wt. %, more preferably 1 to 10 wt. %, and particularly 2 to 8 wt. % of fatty alcohol(s) of formula (II),

H₃C—(CH₂)ₖ—CH₂—OH (II),

in which k represents integers from 4 to 28, preferably 6 to 24, more preferably 8 to 22, and particularly 10, 12, 14, 16, 18, or 20.

Especially preferred hair treatment agents according to the invention include, with respect to the weight of the agent, 0.1 to 20 wt. %, preferably 0.5 to 15 wt. %, more preferably 1 to 10 wt. %, and particularly 2 to 8 wt. % of alcohol(s) from the group consisting of 1-dodecanol (lauryl alcohol), 1-hexadecanol (cetyl alcohol), 1-octadecanol (stearyl alcohol), and/or 1-docosanol (behenyl alcohol), wherein the amounts relate to the total amount of the mentioned fatty alcohols in the composition.

Exceedingly preferred hair treatment agents according to the invention include, with respect to the weight of the agent, 0.1 to 20 wt. %, preferably 0.5 to 15 wt. %, more preferably 1 to 10 wt. %, and particularly 2 to 8 wt. % of alcohol(s) from the group consisting of 1-hexadecanol (cetyl alcohol), and 1-octadecanol (stearyl alcohol), wherein the amounts relate to the total amount of the mentioned fatty alcohols in the composition.

The hair treatment agents include at least one cationic surfactant.

The one or more cationic surfactants are preferably selected from the group of the quaternary ammonium compounds and/or the amidoamines, wherein preferred cationic surfactant(s) is/are selected from: alkyltrimethylammonium chlorides having preferably 10 to 18 carbon atoms in the alkyl residue; dialkyldimethylammonium chlorides having preferably 10 to 18 carbon atoms in the alkyl residue; trialkylmethylammonium chlorides having preferably 10 to 18 carbon atoms in the alkyl residue; cetyltrimethylammonium chloride; stearyltrimethylammonium chloride; behenyltrimethylammonium chloride; distearyldimethylammonium chloride; lauryldimethylammonium chloride; lauryldimethylbenzylammonium chloride; tricetylmethylammonium chloride; Quaternium-27; and/or Quaternium-83.

Hair treatment agents preferred according to the invention include, with respect to the weight of the agent, 0.05 to 20 wt. %, preferably 0.1 to 10 wt. %, more preferably 0.25 to 8 wt. %, and particularly 0.5 to 7 wt. % of cationic surfactant(s).

Especially preferred cationic surfactants are selected from compounds of the following formula (I),

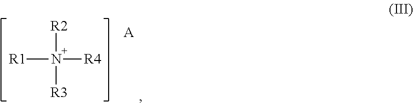

in which at most three residues R1 to R4 represent, independently of each other, a saturated or unsaturated, branched or unbranched alkyl group having 1 to 4 carbon atoms, at least one residue R1 to R4 represents a saturated or unsaturated, branched or unbranched alkyl chain having 8 to 30 carbon atoms, and A means a physiologically acceptable organic or inorganic anion.

In preferred compounds according to formula (I): two or three residues R1 to R4 represent a methyl group or an ethyl group; one or two residues R1 to R4 represent a saturated or unsaturated, branched or unbranched alkyl chain having 14 to 26 carbon atoms; and A represents a halide ion, a sulfate ion of the general formula RSO₃⁻, in which R has the meaning of a saturated or unsaturated alkyl residue having 1 to 4 carbon atoms, or an anionic residue of an organic acid such as maleic acid, fumaric acid, oxalic acid, tartaric acid, citric acid, lactic acid, or acetic acid.

More preferred are compounds according to formula (III), in which: three residues R1 to R4 represent methyl groups; one residue R1 to R4 represents a cetyl, palmityl, stearyl, arachidyl, or behenyl group; and A represents a chloride or a methosulfate ion.

The at least one compound according to formula (I) is especially preferably selected from cetyltrimethylammonium chloride, cetyltrimethylammonium methosulfate, behenyltrimethylammonium chloride, and/or behentrimethylammonium methosulfate. Said compounds can be used in the agent according to the invention individually or in combination, wherein the total amount of compounds of formula (I) in the agent is preferably at most 10 wt. %, and wherein the amount specification relates to the total weight of the agent according to the invention.

An agent according to the invention particularly preferably includes behentrimethylammonium chloride as a cationic surfactant. Here, hair treatment agents according to the invention that include 0.05 to 20 wt. %, preferably 0.1 to 10 wt. %, more preferably 0.25 to 8 wt. %, and particularly 0.5 to 7 wt. % of behenyltrimethylammonium chloride with respect to the weight of said hair treatment agents are preferred.

The hair treatment agents can also include at least one esterquat as a cationic surfactant.

In the sense of the present invention, the term “esterquats” is preferably understood to mean compounds of the following formula (IV),

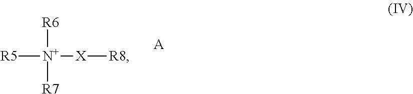

in which, the residues R5, R6, and R7, independently of each other, can be the same of different and have the following meaning: a saturated or unsaturated, branched or unbranched alkyl residue having 1 to 4 carbon atoms, which alkyl residue can include at least one hydroxyl group; a saturated or unsaturated, branched or unbranched alkyl residue or a cyclic saturated or unsaturated alkyl residue having 6 to 30 carbon atoms, which alkyl residue can include at least one hydroxyl group; an aryl or an alkylaryl residue, such as phenyl or benzyl; or (—X—R8), with the stipulation that at most 2 of the residues R5, R6, and R7 can represent (—X—R8), wherein X has the following meaning: —(CH₂)ₙ—, with n=1 to 20, preferably n=1 to 10, and especially preferably n=1-5; —(CH₂—CHR9-O)ₙ—, with n=1 to 200, preferably 1 to 100, more preferably 1 to 50, and especially preferably 1 to 20, and with R9 meaning hydrogen, methyl, or ethyl; or a hydroxyalkylene group having one to four carbon atoms, which hydroxyalkylene group can be branched or unbranched and includes at least one and at most three hydroxyl groups, and wherein R8 has the following meaning: R10-O—CO—, in which R10 is a saturated or unsaturated, branched or unbranched alkyl residue or a cyclic, saturated or unsaturated alkyl residue having 6 to 30 carbon atoms, which alkyl residue can include at least one hydroxyl group and possibly can also be oxethylated with 1 to 100 ethylene oxide units and/or 1 to 100 propylene oxide units; or R11-CO—, in which R11 is a saturated or unsaturated, branched or unbranched alkyl residue or a cyclic, saturated or unsaturated alkyl residue having 6 to 30 carbon atoms, which alkyl residue can include at least one hydroxyl group and possibly can also be oxethylated with 1 to 100 ethylene oxide units and/or 1 to 100 propylene oxide units, and in which A represents a physiologically acceptable organic or inorganic anion. Preferably, one of the residues R5, R6, or R7 represents the group (—X—R8), R8 represents a non-ethoxylated fatty acid residue, such as a palmitic, stearic, arachidic, or behenic acid residue, particularly a stearic acid residue, and A represents a halogenide ion, a sulfate ion of the general formula RSO₃⁻, in which R has the meaning of a saturated or unsaturated alkyl residue having 1 to 4 carbon atoms, or an anionic residue of an organic acid such as maleic acid, fumaric acid, oxalic acid, tartaric acid, citric acid, lactic acid, or acetic acid, particularly for a chloride ion or a methosulfate ion.

The esterquats suitable for the agents according to the invention are preferably selected from at least one of the products sold under the trade names Rewoquat®, Stepantex®, Dehyquart®, Armocare®, and Akypoquat®. Specific examples of esterquats especially suitable according to the invention are the products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90, and Akypoquat® 131.

Especially preferred are agents according to the invention that include as an esterquat at least one of the compounds known under the INCI names Distearoylethyl Hydroxyethylmonium Methosulfate and Distearoylethyl Hydroxyethylmonium Chloride.

Particularly preferred is Distearoylethyl Hydroxyethylmonium Methosulfate, which can be included in the agents according to the invention in a preferred amount of 0.1 to 10 wt. %, more preferably 0.5 to 8 wt. %, especially preferably 0.75 to 6 wt. %, and particularly 1 to 5 wt. %, wherein the amount specifications relate to the total weight of the agent according to the invention.

The one or more esterquats can be added to the agents according to the invention individually or as a mixture with other care active substances.

Because the better manageability and processability, it can be advantageous if the one or more esterquats, particularly Distearoylethyl Hydroxyethylmonium Methosulfate, is/are added to the agents according to the invention as an active-substance mixture. An especially suitable example of such an active-substance mixture is available, for example, from BASF under the trade name Dehyquart® F 75 (Distearoylethyl Hydroxyethylmonium Methosulfate and Cetearyl Alcohol).

The hair treatment agents include at least one silicone.

Agents preferred according to the invention are characterized in that said agents include at least one silicone selected from: (i) polyalkylsiloxanes, polyarylsiloxanes, polyalkylarylsiloxanes that are volatile or non-volatile, straight-chained, branched, or cyclic, cross-linked or uncross-linked; (ii) polysiloxanes that include, in the general structure thereof, one or more organofunctional groups selected from: substituted or unsubstituted aminated groups, (per)fluorinated groups, thiol groups, carboxylate groups, hydroxylated groups, alkoxylated groups, acyloxyalkyl groups, amphoteric groups, bisulfite groups, hydroxyacylamino groups, carboxy groups, sulfonic acid groups, and/or sulfate or thiosulfate groups; (iii) linear polysiloxane (A)-polyoxyalkylene (B) block copolymers of the type (A-B)ₙ with n>3; (iv) graft silicone polymers having an organic backbone that does not include silicone, which graft silicone polymers consist of an organic main chain that is formed of organic monomers that do not include any silicone, onto which main chain at least one polysiloxane macromer has been grafted in the chain and possibly at at least one chain end; (v) graft silicone polymers having a polysiloxane backbone onto which organic monomers not including silicone have been grafted, which graft silicone polymers have a polysiloxane main chain, onto which at least one organic macromer including no silicone has been grafted in the chain and possibly at at least one of the ends of the polysiloxane main chain; and/or mixtures thereof.

Hair treatment agents preferred according to the invention are characterized in that said hair treatment agents include, with respect to the weight of the agent, 0.01 to 20 wt. %, preferably 0.1 to 10 wt. %, more preferably 0.5 to 7.5 wt. %, and particularly 1 to 5 wt. % of silicone(s).

Preferred silicones are described below.

Especially preferred agents according to the invention are characterized in that said agents include at least one silicone of formula Si—I,

(CH₃)₃Si—[O—Si(CH₃)₂]ₓ—O—Si(CH₃)₃ (Si-I),

in which x represents a number from 0 to 100, preferably 0 to 50, more preferably 0 to 20, and particularly 0 to 10.

These silicones are called DIMETHICONE according to the INCI nomenclature. In the context of the present invention, the following compounds are preferably used as a silicone of formula Si-I:
(CH₃)₃Si—O—Si(CH₃)₃(CH₃)₃Si—O—(CH₃)₂Si—O—Si(CH₃)₃,
(CH₃)₃Si—[O—(CH₃)₂Si]₂—O—Si(CH₃)₃,
(CH₃)₃Si—[O—(CH₃)₂Si]₃—O—Si(CH₃)₃,
(CH₃)₃Si—[O—(CH₃)₂Si]₄—O—Si(CH₃)₃,
(CH₃)₃Si—[O—(CH₃)₂Si]₅—O—Si(CH₃)₃,
(CH₃)₃Si—[O—(CH₃)₂Si]₆—O—Si(CH₃)₃,
(CH₃)₃Si—[O—(CH₃)₂Si]₇—O—Si(CH₃)₃,
(CH₃)₃Si—[O—(CH₃)₂Si]₈—O—Si(CH₃)₃,
(CH₃)₃Si—[O—(CH₃)₂Si]₉—O—Si(CH₃)₃,
(CH₃)₃Si—[O—(CH₃)₂Si]₁₀—O—Si(CH₃)₃,
(CH₃)₃Si—[O—(CH₃)₂Si]₁₁—O—Si(CH₃)₃,
(CH₃)₃Si—[O—(CH₃)₂Si]₁₂—O—Si(CH₃)₃,
(CH₃)₃Si—[O—(CH₃)₂Si]₁₃—O—Si(CH₃)₃,
(CH₃)₃Si—[O—(CH₃)₂Si]₁₄—O—Si(CH₃)₃,
(CH₃)₃Si—[O—(CH₃)₂Si]₁₅—O—Si(CH₃)₃,
(CH₃)₃Si—[O—(CH₃)₂Si]₁₆—O—Si(CH₃)₃,
(CH₃)₃Si—[O—(CH₃)₂Si]₁₇—O—Si(CH₃)₃,
(CH₃)₃Si—[O—(CH₃)₂Si]₁₈—O—Si(CH₃)₃,
(CH₃)₃Si—[O—(CH₃)₂Si]₁₉—O—Si(CH₃)₃, and/or
(CH₃)₃Si—[O—(CH₃)₂Si]₂₀—O—Si(CH₃)₃.
With (CH₃)₃Si—O—Si(CH₃)₃, (CH₃)₃Si—O—(CH₃)₂Si—O—Si(CH₃)₃, and/or (CH₃)₃Si—[O—(CH₃)₂Si]₂—O—Si(CH₃)₃ is especially preferred dimethicones.

Of course, mixtures of the aforementioned silicones can also be included in the agents according to the invention. Preferred silicones that can be used according to the invention have viscosities of 0.2 to 2 mm²s⁻¹ at 20° C., wherein silicones having viscosities of 0.5 to 1 mm²s⁻¹ are especially preferred.

Especially preferred agents according to the invention include one or more amino-functional silicones. Such silicones can be described, for example, by the formula

M(RₐQ_{b}SiO_{(4-a-b)/2})ₓR_{c}SiO_{(4-c)/2})_{y}M,

wherein in the formula above R is a hydrocarbon or a hydrocarbon residue having 1 to approximately 6 carbon atoms, Q is a polar residue of the general formula -—R¹HZ, in which R¹ is a divalent, linking group that is bonded to hydrogen and to the residue Z and composed of carbon and hydrogen atoms, carbon, hydrogen, and oxygen atoms, or carbon, hydrogen, and nitrogen atoms, and Z is an organic, amino-functional residue that includes at least one amino-functional group; “a” assumes values in the range of approximately 0 to approximately 2, “b” assumes values in the range of approximately 1 to approximately 3, “a”+“b” is less than or equal to 3, and “c” is a number in the range of approximately 1 to approximately 3, and x is a number in the range of 1 to approximately 2,000, preferably approximately 3 to approximately 50, and most preferably approximately 3 to approximately 25, and y is a number in the range of approximately 20 to approximately 10,000, preferably approximately 125 to approximately 10,000, and most preferably approximately 150 to approximately 1,000, and M is a suitable silicone end group known in the prior art, preferably trimethylsiloxy. Non-restricting examples of the residues represented by R include alkyl residues, such as methyl, ethyl, propyl, isopropyl, isopropyl, butyl, isobutyl, amyl, isoamyl, hexyl, isohexyl, and the like; alkenyl residues, such as vinyl, halovinyl, alkylvinyl, allyl, haloallyl, alkylallyl; cycloalkyl residues, such as cyclobutyl, cyclopentyl, cyclohexyl, and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl, and the like; and sulfur-including residues, such as mercaptoethyl, mercaptopropyl, mercaptohexyl, mercaptophenyl, and the like. R is preferably an alkyl residue including 1 to approximately 6 carbon atoms. R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—, and —(CH₂)₃C(O)SCH₂CH₂—.

Z is an organic, amino-functional residue including at least one functional amino group. A possible formula for Z is NH(CH₂)_{z}NH₂, in which z is 1 or more. Another possible formula for Z is —NH(CH₂)_{z}(CH₂)_{zz}NH, in which both z and zz are, independently, 1 or more, wherein this structure includes diamino ring structures, such as piperazinyl. Z is most preferably an —NHCH₂CH₂NH₂ residue. Another possible formula for Z is —N(CH₂)_{z}(CH₂)_{zz}NX₂ or —NX₂, in which each X of X₂ is selected independently from the group consisting of hydrogen and alkyl groups having 1 to 12 carbon atoms and zz is 0.

Q is most preferably a polar, amino-functional residue of the formula —CH₂CH₂CH₂NHCH₂CH₂NH₂. In the formulas, “a” assumes values in the range of approximately 0 to approximately 2, “b” assumes values in the range of approximately 2 to approximately 3, “a”+“b” is less than or equal to 3, and “c” is a number in the range of approximately 1 to approximately 3. The molar ratio of the RₐQ_{b}SiO_{(4-a-b)/2} units to the R_{c}SiO_{(4-c)/2} units lies in the range of approximately 1:2 to 1:65, preferably approximately 1:5 to approximately 1:65, and most preferably approximately 1:15 to approximately 1:20. If one or more silicones of the formula above are used, the various variable substituents in the formula above can be different in the case of the various silicone components present in the silicone mixture.

Preferred agents according to the invention are characterized in that said agents include an amino-functional silicone of formula (Si-II),

R′ₐG₃₋ₐ-Si(OSiG₂)ₙ-(OSiG_{b}R′_{2-b})ₘ—O—SiG₃₋ₐ-R′ₐ (Si-II),

in which: G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, and/or —C(CH₃)₃, a is a number between 0 and 3, particularly 0; b is a number between 0 and 1, particularly 1; m and n are numbers having a sum (m +n) between 1 and 2000, preferably between 50 and 150, wherein n preferably assumes value of 0 to 1999, particularly 49 to 149, and m preferably assumes values of 1 to 2000, particularly 1 to 10; R′ is a monovalent residue selected from: -Q-N(R″)—CH₂—CH₂—N(R″)₂, -Q-N(R″)₂, -Q-N⁺(R″)₃A⁻, -Q-N⁺H(R″)₂A⁻, -Q-N⁺H₂(R″)A⁻, and/or -Q-N(R″)—CH₂—CH₂—N⁺R″H₂A⁻, wherein each Q represents a chemical bond, —CH₂—, —CH₂—CH₂—, —CH₂CH₂CH₂—, —C(CH₃)₂—, —CH₂CH₂CH₂CH₂—, —CH₂C(CH₃)₂—, or —CH(CH₃)CH₂CH₂—, and R″ represents identical or different residues from the group including —H, -phenyl, -benzyl, —CH₂—CH(CH₃)Ph, the C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, and —C(CH₃)₃, and A represents an anion, which is preferably selected from chloride, bromide, iodide, or methosulfate.

Especially preferred agents according to the invention are characterized in that said agents include at least one amino-functional silicone of formula (Si-IIa),

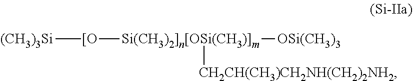

in which m and n are numbers having a sum (m+n) between 1 and 2000, preferably between 50 and 150, wherein n preferably assumes a value of 0 to 1999, particularly 49 to 149, and m preferably assumes a value of 1 to 2000, particularly 1 to 10.

These silicones are called Trimethylsilylamodimethicone according to the INCI declaration.

Also especially preferred are agents according to the invention that include an amino-functional silicone of formula (Si-IIb),

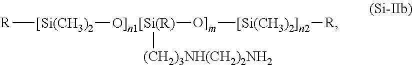

in which R represents —OH, —O—CH₃, or a —CH3 group and m, n1, and n2 are numbers having a sum (m+n1+n2) between 1 and 2000, preferably between 50 and 150, wherein the sum (n1+n2) preferably assumes values of 0 to 1999, particularly 49 to 149, and m preferably assumes values of 1 to 2000, particularly 1 to 10.

These silicones are called Amodimethicone according to the INCI declaration.

Regardless of which amino-functional silicones are used, agents according to the invention that include an amino-functional silicone having an amine value above 0.25 meq/g, preferably above 0.3 meq/g, and particularly above 0.4 meq/g are preferred. The amine value represents the milliequivalents of amine per gram of the amino-functional silicone. The amine value can be determined by titration and can also be stated in the unit mg KOH/g.

Hair treatment agents preferred according to the invention are characterized in that said hair treatment agents include, with respect to the weight thereof, 0.01 to 20 wt. %, preferably 0.1 to 10 wt. %, more preferably 0.5 to 7.5 wt. %, and particularly 1 to 5 wt. % of amino-functional silicone(s).

The agents according to the invention especially preferably include amino-functional silicone(s) having one or more terminal hydroxy groups. Some special amino-functional silicones having one or more terminal hydroxy groups have been found to be especially suitable in the agents according to the invention. These are described below.

Agents preferred according to the invention are characterized in that said agents include, with respect to the weight thereof, 0.01 to 20 wt. %, preferably 0.1 to 10 wt. %, especially preferably 0.5 to 7.5 wt. %, and particularly 1 to 5 wt. % of at least one silicone of formula (Si-III) below,

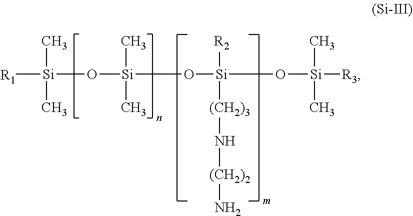

in which: m and n are numbers such that that the sum (n+m) lies in the range of 1 to 1000, n is a number in the range of 0 to 999, and m is a number in the range of 1 to 1000; and R1, R2, and R3, which may be identical or different, are selected from a hydroxy group or a C1-4 alkoxy group and wherein at least one of the groups R1 to R3 is a hydroxy group.

Other agents preferred according to the invention are characterized in that said agents include, with respect to the weight thereof, 0.01 to 20 wt. %, preferably 0.1 to 10 wt. %, especially preferably 0.5 to 7.5 wt. %, and particularly 1 to 5 wt. % of at least one silicone of formula (Si-IV) below,

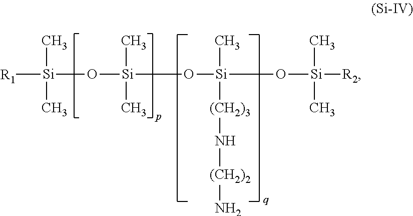

in which: p and q are numbers such that the sum (p+q) lies in the range of 1 to 1000, p is a number in the range of 0 to 999, and q is a number in the range of 1 to 1000; and R1 and R2, which are different, mean a hydroxy group or a C1-4 alkoxy group, wherein at least one of the groups R1 to R2 means a hydroxy group.

The silicones of formulas (Si-III) and (Si-IV) are distinguished by the grouping on the Si atom that bears the nitrogen-including group: R2 means a hydroxy group or a C1-4 alkoxy group in formula (Si-III), while the residue in formula (Si-IV) is a methyl group. The individual Si groupings, which are marked with the indices m and n or p and q, do not have to exist as blocks, but rather the individual units may also be statistically distributed, i.e., each R1-Si(CH₃)₂ group does not necessarily have to be bonded to an —[O—Si(CH₃)₂] grouping in formulas (Si-III) and (Si-IV).

In the method according to the invention, pretreatment agents including at least one silicone of formula (Si-V) have proven especially effective with regard to the desired effects:

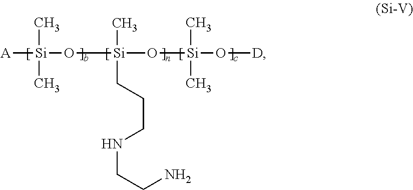

in which: A represents a group selected from: —OH, —O—Si(CH₃)₃, —O—Si(CH₃)₂OH, and —O—Si(CH₃)₂OCH₃, D represents a group selected from: —H, —Si(CH₃)₃, —Si(CH₃)₂OH, and —Si(CH₃)₂OCH₃, b, n, and c are integers between 0 and 1000 such that n>0 and b+c>0; and A=—OH and/or D=—H.

Therefore, hair treatment agents according to the invention that include, with respect to the weight of the agent, 0.01 to 20 wt. %, preferably 0.1 to 10 wt. %, especially preferably 0.5 to 7.5 wt. %, and particularly 1 to 5 wt. % of at least one silicone of formula (Si-V):

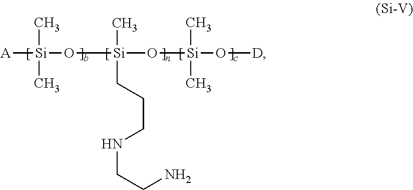

in which: A represents a group selected from: —OH, —O—Si(CH₃)₃, —O—Si(CH₃)₂OH, and —O—Si(CH₃)₂OCH₃, D represents a group selected from: —H, —Si(CH₃)₃, —Si(CH₃)₂OH, and —Si(CH₃)₂OCH₃, b, n, and c are integers between 0 and 1000, such that n>0 and b+c>0; and A=—and/or D=—H, are preferred according to the invention.

In formula (Si-V) stated above, the individual siloxane units having the indices b, c, and n are statistically distributed, i.e., these silicones need not be block copolymers.

Other especially suitable silicones are 4-morpholinomethyl-substituted. Hair treatment agents according to the invention that include, with respect to the weight thereof, 0.01 to 20 wt. %, preferably 0.1 to 10 wt. %, especially preferably 0.5 to 7.5 wt. %, and particularly 1 to 5 wt. % of at least one 4-morpholinomethyl-substituted silicone of formula (Si-VI),

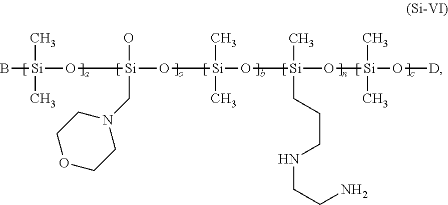

in which: A represents a structural unit (i) bonded by means of an —O,

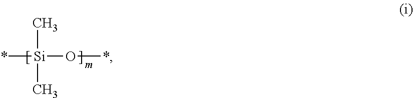

an oligomeric or polymeric residue bonded by means of an —O and including structural units of formula (i), or —OH; * represents a bond to the structural unit (i) or an end group B (Si-bonded) or D (O-bonded); B represents a group selected from: —OH, —O—Si(CH₃)₃, —O—Si(CH₃)₂OH, and —O—Si(CH₃)₂OCH₃, D represents a group selected from: —H, —Si(CH₃)₃, —Si(CH₃)₂OH, and —Si(CH₃)₂OCH₃, a, b, and c are integers between 0 and 1000, such that a+b+c>0; m, n, and o are integers between 1 and 1000; and B=—OH and/or D=—H, are especially preferred.

Structural formula (Si-VI) should make clear that the siloxane groups n and o do not necessarily have to be bonded directly to an end grouping B or D. Rather, a>0 or b>0 in preferred formulas (Si-VI) and a>0 and b>0 in especially preferred formulas (Si-VI), i.e., the terminal grouping B or D is preferably bonded to a dimethylsiloxy grouping. Also in formula (Si-VI), the siloxane units a, b, c, n, and o are preferably statistically distributed.

The silicones used according to the invention represented by formula (Si-VI) can be trimethylsilyl-terminated (D or B=—Si(CH₃)₃). Said silicones can also be dimethylsilylhydroxy-terminated at both ends or dimethylsilylhydroxy-terminated and dimethylsilylmethoxy-terminated at one end. Silicones especially preferably used in the context of the present invention are selected from silicones in which:
B=—O—Si(CH₃)₂OH and D=—Si(CH₃)₃;
B=—O—Si(CH₃)₂OH and D=—Si(CH₃)₂OH;
B=—O—Si(CH₃)₂OH and D=—Si(CH₃)₂OCH₃;
B=—O—Si(CH₃)₃ and D=—Si(CH₃)₂OH; and/or
B=—O—Si(CH₃)₂OCH₃ and D=—Si(CH₃)₂OH
   These silicones lead to great improvements in the hair properties of the hair treated with the agents according to the invention, particularly to greatly improved protection in the case of oxidative treatment.

Also in formula (Si-VI), the residue A can represent: a structural unit (i) bonded by means of an —O; an oligomeric or polymeric residue bonded by means of an —O and including structural units of formula (i); or —OH.

Therefore, formula (Si-VI) is stated more precisely as one of formulas (Si-VIa), (Si-VIb), and/or (Si-VIc):

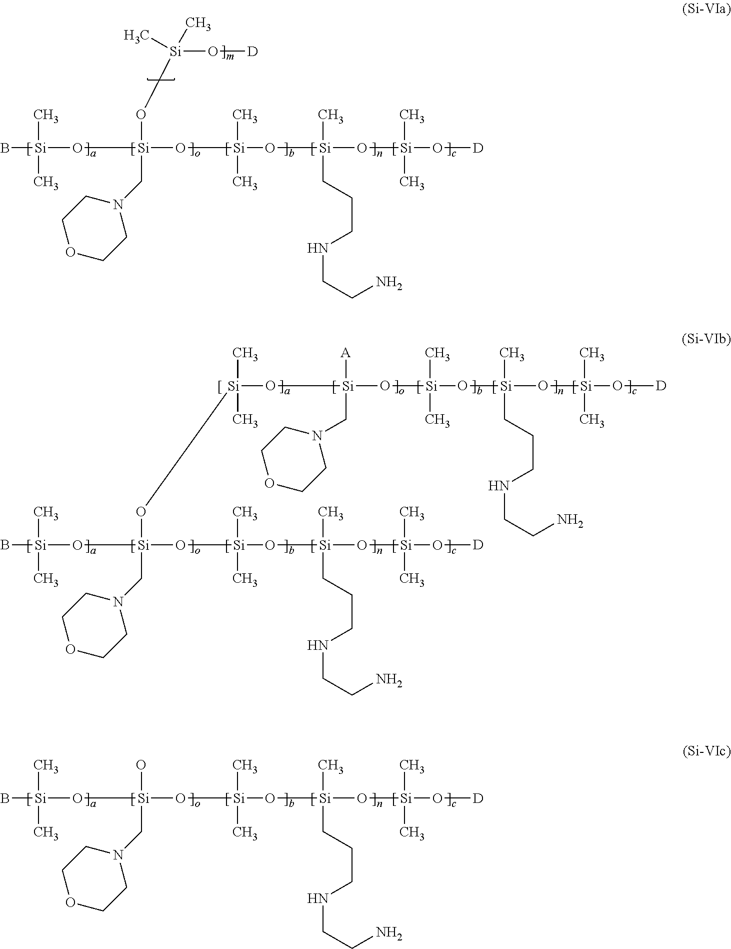

Regardless of the type of the one or more utilized amino-functional silicones having one or more terminal hydroxy groups, the agents according to the invention preferably include the one or more silicones in the form of an emulsion, especially preferably in the form of a microemulsion.

It has been found that the effect of the silicones used in the agents according to the invention can be increased further if certain non-ionic components are likewise used in the agents. In addition, said non-ionic components have positive effects on the storage stability of the agents. Non-ionic components that are especially suitable here are ethoxylates of decanol, undecanol, dodecanol, tridecanol, etc. Ethoxylated tridecanols have proven especially suitable and are especially preferably incorporated into the agents according to the invention. Agents especially preferred according to the invention are characterized in that said agents include, with respect to the weight of the agent, 0.00001 to 5 wt. %, preferably 0.0001 to 3.5 wt. %, especially preferably 0.001 to 2 wt. %, more preferably 0.01 to 1 wt. %, and particularly 0.1 to 0.5 wt. % of branched, ethoxylated tridecanol (INCI name: Trideceth-5) or α-iso-tridecyl-ω-hydroxy polyglycol ether (INCI name: Trideceth-10) or mixtures thereof.

The agents according to the invention include 10-undecenoic acid (undecylenic acid) and/or salts of undecylenic acid.

10-Undecenoic acid is preferably used, wherein use within certain narrow amount ranges is especially effective. Preferred hair treatment agents according to the invention include, with respect to the weight of the agent, 0.001 to 1 wt. %, preferably 0.01 to 0.5 wt. %, more preferably 0.05 to 0.3 wt. %, and particularly 0.1 to 0.2 wt. % of 10-undecenoic acid.

In addition to or in place of 10-undecenoic acid, salts of 10-undecenoic acid may also be used. Here, the alkali-metal salts, sodium undecylenate and potassium undecylenate, ammonium undecylenate, magnesium undecylenate, calcium undecylenate, zinc undecylenate, iron undecylenate, and manganese undecylenate, among others, have proven especially suitable. Preferred hair treatment agents according to the invention include salt(s) from the group of sodium undecylenate, potassium undecylenate, ammonium undecylenate, magnesium undecylenate, calcium undecylenate, zinc undecylenate, iron undecylenate, and manganese undecylenate in a total amount of 0.001 to 0.4 wt. %, preferably 0.01 to 0.3 wt. %, more preferably 0.025 to 0.25 wt. %, and particularly 0.05 to 0.2 wt. % with respect to the weight of the agent.

Exceedingly preferred hair treatment agents include, with respect to the weight of the agent, 0.001 to 1 wt. %, preferably 0.01 to 0.5 wt. %, more preferably 0.05 to 0.3 wt. %, and particularly 0.1 to 0.2 wt. % of salt(s) from the group of ammonium undecylenate, sodium undecylenate, potassium undecylenate, magnesium undecylenate, calcium undecylenate, zinc undecylenate, and manganese undecylenate.

In one example, the hair treatment agents include, with respect of the weight of the agent, 0.001 to 0.4 wt. %, preferably 0.01 to 0.3 wt. %, more preferably 0.025 to 0.25 wt. %, and particularly 0.05 to 0.2 wt. % of sodium ortho-phenylphenolate.

The hair treatment agents include the active substances described above preferably in a cosmetically acceptable carrier. In the context of the invention, the term “cosmetically acceptable carrier” is preferably understood to mean an aqueous or aqueous-alcoholic carrier.

The cosmetic carrier includes preferably at least 50 wt. %, more preferably at least 60 wt. %, especially preferably at least 70 wt. %, and particularly preferably at least 75 wt. % of water. Furthermore, the cosmetic carrier can include 0.01 to 40 wt. %, preferably 0.05 to 30 wt. %, and particularly 0.1 to 20 wt. % of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1, hexanol, 2-hexanol, 1,2-hexanediol, 1,6-hexanediol, polyethylene glycols, sorbitol, sorbitan, benzyl alcohol, or mixtures of these alcohols.

The water-soluble alcohols are especially preferred. Ethanol, 1,2-propylene glycol, glycerol, benzyl alcohol, and mixtures of these alcohols are particularly preferred.

For very good (scalp) skin compatibility of the hair treatment agents according to the invention, it is advantageous if said hair treatment agents have a slightly acidic pH value. It has been found that the agents according to the invention have especially good skin compatibility and gentleness in a pH range of 4.2 to 5.8.

In one embodiment, the hair treatment agents according to the invention therefore preferably have a pH value in the range of 4.2 to 5.8, more preferably 4.25 to 5.6, especially preferably 4.3 to 5.5, extraordinarily preferably 4.35 to 5.4, and particularly preferably 4.4 to 5.3.

The hair treatment agents according to the invention can include plant oils, plant butters, and/or plant waxes. These plant oil components give the hair improved combability and manageability and increase the hair shine.

The suitable plant oil components include natural (plant) oils and/or butters, which usually include triglycerides and mixtures of triglycerides.

Preferred natural oils are coconut oil, (sweet) almond oil, walnut oil, peach kernel oil, apricot kernel oil, argan oil, avocado oil, tea tree oil, soy oil, sesame oil, sunflower oil, tsubaki oil, evening primrose oil, rice bran oil, palm kernel oil, mango kernel oil, marula oil, meadowfoam oil, safflower oil, macadamia nut oil, grape seed oil, amaranth seed oil, bamboo oil, olive oil, wheat germ oil, pumpkin seed oil, mallow oil, hazelnut oil, safflower seed oil, canola oil, sasanqua oil, jojoba oil, rambutan oil, cocoa butter, and/or shea butter.

Carnauba wax, beeswax, and/or candelilla wax can be used with preference as suitable natural or plant waxes.

Especially preferred plant oil components are (sweet) almond oil, peach kernel oil, apricot kernel oil, amaranth seed oil, argan oil, olive oil, jojoba oil, cocoa butter, and/or shea butter. Apricot kernel oil, argan oil, olive oil, and/or jojoba oil is particularly preferred.

In a preferred embodiment, the hair treatment agents according to the invention preferably include coconut oil, (sweet) almond oil, walnut oil, peach kernel oil, apricot kernel oil, argan oil, avocado oil, tea tree oil, soy oil, sesame oil, sunflower oil, tsubaki oil, evening primrose oil, rice bran oil, palm kernel oil, mango kernel oil, marula oil, meadowfoam oil, safflower oil, macadamia nut oil, grape seed oil, amaranth seed oil, bamboo oil, olive oil, wheat germ oil, pumpkin seed oil, mallow oil, hazelnut oil, safflower seed oil, canola oil, sasanqua oil, jojoba oil, rambutan oil, cocoa butter, and/or shea butter.

Within this embodiment, it is especially preferred if the hair treatment agents according to the invention include (sweet) almond oil, peach kernel oil, apricot kernel oil, amaranth seed oil, argan oil, olive oil, jojoba oil, cocoa butter, and/or shea butter.

The weight percentage of the at least one plant oil, the plant butter, and/or the plant wax with respect to the total weight of the hair treatment agents according to the invention is preferably 0.02 to 2.50 wt. %, more preferably 0.03 to 2.00 wt. %, especially preferably 0.04 to 1.50 wt. %, and particularly 0.05 to 1.00 wt. %.

In another preferred embodiment, the hair treatment agents according to the invention include, in addition to the previously mentioned essential and optional constituents, at least one further hair-conditioning active substance selected from the group including the following in order to further increase the care properties of the agents: protein hydrolysates, vitamins, plant extracts, and/or glycerol.

Product mixtures that can be obtained by the acidically, basically, or enzymatically catalyzed decomposition of proteins should be understood by suitable protein hydrolysates.

Protein hydrolysates of plant, animal, and/or marine origin can be used.

Animal protein hydrolysates are, for example, protein hydrolysates of elastin, collagen, keratin, silk, and milk protein, which can also be present in the form of salts. Such products are sold, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Protein hydrolysates of plant origin, e.g., protein hydrolysates of soy, almond, rice, pea, potato, and wheat. Such products are available, for example, under the trademarks Gluadin® (Cognis), DiaMin® (Diamalt), Lexein® (Inolex), and Crotein® (Croda). Cationized protein hydrolysates can also be used, wherein the protein hydrolysate used as a basis can originate from animals, for example from collagen, milk, or keratin, from plants, for example from wheat, corn, rice, potatoes, soy, or almonds, from marine lifeforms, for example from fish collagen or algae, or from biotechnologically obtained protein hydrolysates. The protein hydrolysates on which the cationic derivatives are based can be obtained from the corresponding proteins by chemical hydrolysis, particularly alkaline or acidic hydrolysis, by enzymatic hydrolysis, and/or by a combination of the two hydrolysis types. The hydrolysis of proteins generally results in a protein hydrolysate having a molecular weight distribution from approximately 100 daltons to several thousand daltons. Cationic protein hydrolysates based on a protein fraction having a molecular weight of 100 to 25000 daltons, preferably 250 to 5000 daltons, are preferred. Furthermore, quaternized amino acids and mixtures thereof should be understood under the term “cationic protein hydrolysates”. The quaternization of the protein hydrolysates or the amino acids is often performed by means of quaternary ammonium salts, such as N,N-dimethyl-N-(n-alkyl)-N-(2-hydroxy-3-chloro-n-propyl)-ammonium halides. Furthermore, the cationic protein hydrolysates can also be further derivatized. The following commercially available products known under the respective INCI names are mentioned as typical examples of the cationic protein hydrolysates and protein hydrolysate derivatives: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium

Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The weight percentage of the one or more protein hydrolysates with respect to the total weight of the hair treatment agents is preferably 0.01 to 5 wt. %, more preferably 0.025 to 3 wt. %, and particularly 0.05 to 2 wt. %.

Regardless of the source (plant, animal, marine, etc.), protein hydrolysates include individual amino acids, oligopeptides, and possibly polypeptides in accordance with the degree of hydrolysis.

The hair treatment agents according to the invention especially preferably include at least one oligopeptide that has at least one amino acid sequence Glu-Glu-Glu, wherein the amino group can be free or protonated and the carboxy groups can be free or deprotonated.

Preferred hair treatment agents according to the invention are characterized in that said hair treatment agents include, with respect to the weight of the agent, 0.0001 to 10 wt. % of at least one oligopeptide that has at least one amino acid sequence Glu-Glu-Glu, wherein the amino group can be free or protonated and the carboxy groups can be free or deprotonated.

In this formula as in all following formulas, the bracketed hydrogen atom (H) of the amino group and the bracketed hydroxy group (OH) of the acid function mean that the groups in question can be present as such (in which case the oligopeptide in question is an oligopeptide having the particular number of amino acids as shown (in formula 3 above)) or that the amino acid sequence is present in an oligopeptide that also includes additional amino acids—depending on where the one or more additional amino acids are bonded, the bracketed components of the aforementioned formula are replaced by the one or more additional amino acid residues.

These preferred hair treatment agents according to the invention include, with respect to the weight of the agent, 0.0001 to 10 wt. % of at least one oligopeptide that has at least one amino acid sequence Glu-Glu-Glu, i.e., at least three consecutive glutamic acids.

In the sense of the present application, oligopeptides are condensation products of amino acids, which condensation products are linked by peptide bonds in the manner of an acid amide and include at least 3 and at most 25 amino acids.

In preferred hair treatment agents according to the invention, the oligopeptide includes 5 to 15 amino acids, preferably 6 to 13 amino acids, especially preferably 7 to 12 amino acids, and particularly 8, 9, or 10 amino acids.

The molar mass of the oligopeptide included in the agents according to the invention can vary depending on whether additional amino acids are bonded to the sequence Glu-Glu-Glu and depending on the type of said amino acids. Preferred hair treatment agents used according to the invention are characterized in that the oligopeptide has a molar mass of 650 to 3000 daltons, preferably 750 to 2500 daltons, especially preferably 850 to 2000 daltons, and particularly 1000 to 1600 daltons.

In summary, preferred hair treatment agents are characterized in that the oligopeptide includes 5 to 15 amino acids, preferably 6 to 13 amino acids, especially preferably 7 to 12 amino acids, and particularly 8, 9, or 10 amino acids and has a molar mass of 650 to 3000 daltons, preferably 750 to 2500 daltons, especially preferably 850 to 2000 daltons, and particularly 1000 to 1600 daltons.

As can be seen from the preferred number of amino acids in the oligopeptides and the preferred molar mass range, oligopeptides that consist not only of the three glutamic acids but also of additional amino acids bonded to this sequence are preferably used. Said additional amino acids are preferably selected from certain amino acids, while certain other representatives are less preferred according to the invention.

It is preferred if the oligopeptides used in the agents according to the invention do not include methionine. It is more preferred if the oligopeptides used in the agents according to the invention do not include cysteine and/or cystine. It is more preferred if the oligopeptides used in the agents according to the invention do not include aspartic acid and/or asparagine. It is more preferred if the oligopeptides used in the agents according to the invention do not include serine and/or threonine.

In contrast, it is preferred if the oligopeptides used in the agents according to the invention include tyrosine. It is more preferred if the oligopeptides used in the agents according to the invention include leucine. It is more preferred if the oligopeptides used in the agents according to the invention include isoleucine. It is more preferred if the oligopeptides used in the agents according to the invention include arginine. It is more preferred if the oligopeptides used in the agents according to the invention include valine.

Especially preferred oligopeptides and amino acid sequences included in the preferred oligopeptides are described below:

An especially preferred oligopeptide additionally includes tyrosine, which is preferably bonded by means of the acid function thereof to the Glu-Glu-Glu sequence. Therefore, hair treatment agents preferred according to the invention are characterized in that the oligopeptide included therein has at least one amino acid sequence SEQ ID NO:1 Tyr-Glu-Glu-Glu, wherein the amino group can be free or protonated and the carboxy groups can be free or deprotonated.

Another especially preferred oligopeptide additionally includes isoleucine, which is preferably bonded by means of the amino function thereof to the Glu-Glu-Glu sequence. Therefore, hair treatment agents preferred according to the invention are characterized in that the oligopeptide included therein has at least one amino acid sequence SEQ ID NO:2 Glu-Glu-Glu-Ile, wherein the amino group can be free or protonated and the carboxy groups can be free or deprotonated.

Oligopeptides that have both aforementioned amino acids (tyrosine and isoleucine) are preferred according to the invention. Especially preferred are hair treatment agents according to the invention in the case of which the oligopeptide included in the hair treatment agent has at least one amino acid sequence SEQ ID NO:3 Tyr-Glu-Glu-Glu-Ile, wherein the amino group can be free or protonated and the carboxy groups can be free or deprotonated.

More highly preferred oligopeptides additionally include arginine, which is preferably bonded to isoleucine.

Especially preferred are hair treatment agents according to the invention in the case of which the oligopeptide included in the hair treatment agent has at least one amino acid sequence SEQ ID NO:4 Tyr-Glu-Glu-Glu-Ile-Arg, wherein the amino groups can be free or protonated and the carboxy groups can be free or deprotonated.

Even more highly preferred oligopeptides additionally include valine, which is preferably bonded to the arginine. Therefore, hair treatment agents more highly preferred according to the invention are characterized in that the oligopeptide included in the hair treatment agent has at least one amino acid sequence SEQ ID NO:5 Tyr-Glu-Glu-Glu-Ile-Arg-Val, wherein the amino groups can be free or protonated and the carboxy groups can be free or deprotonated.

Even more highly preferred oligopeptides additionally include leucine, which is preferably bonded to the valine. Therefore, hair treatment agents more highly preferred according to the invention are characterized in that the oligopeptide included in the hair treatment agent has at least one amino acid sequence SEQ ID NO:6 Tyr-Glu-Glu-Glu-Ile-Arg-Val-Leu, wherein the amino groups can be free or protonated and the carboxy groups can be free or deprotonated.

Particularly preferred oligopeptides additionally include leucine, which is preferably bonded to the tyrosine. Therefore, hair treatment agents more highly preferred according to the invention are characterized in that the oligopeptide included in the hair treatment agent has at least one amino acid sequence SEQ ID NO:7 Leu-Tyr-Glu-Glu-Glu-Ile-Arg-Val-Leu, wherein the amino groups can be free or protonated and the carboxy groups can be free or deprotonated.

Agents according to the invention exceedingly preferably include at least two oligopeptides that satisfy the aforementioned mentioned criteria but differ from each other. Thus, for example, the use of hair treatment agents that include at least two oligopeptides A and B, which differ from each other and which both include the amino acid sequence Glu-Glu-Glu, is preferred.

Such oligopeptides A and B differing from each other correspond to each other in that said oligopeptides bear three consecutive Glu amino acids in the amino acid sequence of the oligopeptides but have differences in the amino acids bonded before or after the three consecutive Glu amino acids. Peptides differing from each other and having partial correspondence, which can be much greater than in the aforementioned three amino acids, are preferred.

Thus, more highly preferred hair treatment agents are characterized in that at least two oligopeptides A and B, which differ from each other and which both include the amino acid sequence SEQ ID NO:2 Glu-Glu-Glu-Ile, are included in the hair treatment agent.

Likewise preferred are hair treatment agents that include at least two oligopeptides A and B, which differ from each other and which both include the amino acid sequence SEQ ID NO:1 Tyr-Glu-Glu-Glu.

Even more highly preferred hair treatment agents are characterized in that said hair treatment agents include at least two oligopeptides A and B, which differ from each other and which both include the amino acid sequence SEQ ID NO:8 Glu-Glu-Glu-Ile-Arg.

Likewise, even more highly preferred hair treatment agents are characterized in that the hair treatment agent includes at least two oligopeptides A and B, which differ from each other and which both include the amino acid sequence SEQ ID NO:3 Tyr-Glu-Glu-Glu-Ile.

Therefore, preferred hair treatment agents according to the invention are characterized in that the oligopeptide has at least one amino acid sequence Tyr-Glu-Glu-Glu-Ile, wherein the amino group can be free or protonated and the carboxy groups can be free or deprotonated.

Exceedingly preferred hair treatment agents are characterized in that the hair treatment agent includes at least two oligopeptides A and B, which differ from each other and which both include the amino acid sequence SEQ ID NO:8 Glu-Glu-Glu-Ile-Arg.

Likewise, exceedingly preferred hair treatment agents are characterized in that the hair treatment agent includes at least two oligopeptides A and B, which differ from each other and which both include the amino acid sequence SEQ ID NO:4 Tyr-Glu-Glu-Glu-Ile-Arg.

There is preferably even greater structural correspondence in the oligopeptides. Thus, hair treatment agents that include at least two oligopeptides A and B, which differ from each other and which both include the amino acid sequence SEQ ID NO:9 Glu-Glu-Glu-Ile-Arg-Val, are other preferred embodiments of the present invention.

Likewise, preferred embodiments are hair treatment agents that include at least two oligopeptides A and B, which differ from each other and which both include the amino acid sequence SEQ ID NO:5 Tyr-Glu-Glu-Glu-Ile-Arg-Val.

Even more highly preferred hair treatment agents according to the invention are characterized in that said hair treatment agents include at least two oligopeptides A and B, which differ from each other and which both include the amino acid sequence SEQ ID NO:10 Glu-Glu-Glu-Ile-Arg-Val-Leu.

Likewise, even more highly preferred hair treatment agents according to the invention are characterized in that said hair treatment agents include at least two oligopeptides A and B, which differ from each other and which both include the amino acid sequence SEQ ID NO:6 Tyr-Glu-Glu-Glu-Ile-Arg-Val-Leu.

Therefore, preferred hair treatment agents according to the invention are characterized in that the oligopeptide has at least one amino acid sequence SEQ ID NO:6 Tyr-Glu-Glu-Glu-Ile-Arg-Val-Leu, wherein the amino groups can be free or protonated and the carboxy groups can be free or deprotonated.

Particularly preferred hair treatment agents according to the invention are characterized in that said hair treatment agents include at least two oligopeptides A and B, which differ from each other, wherein the oligopeptide A has the amino acid sequence SEQ ID NO:7 Leu-Tyr-Glu-Glu-Glu-Ile-Arg-Val-Leu, wherein the amino groups can be free or protonated and the carboxy groups can be free or deprotonated, and the oligopeptide B has the amino acid sequence SEQ ID NO:6 Tyr-Glu-Glu-Glu-Ile-Arg-Val-Leu, wherein the amino groups can be free or protonated and the carboxy groups can be free or deprotonated.

In exceedingly preferred hair treatment agents of this last-mentioned embodiment, said hair treatment agents include, with respect to the weight of the agent, 0.00001 to 1 wt. % of oligopeptide A and 0.00001 to 1 wt. % of oligopeptide B.

In more highly preferred hair treatment agents of this last-mentioned embodiment, said hair treatment agents include, with respect to the weight of the agent, 0.00005 to 0.1 wt. % of oligopeptide A and 0.00005 to 0.1 wt. % of oligopeptide B.

In even more highly preferred hair treatment agents of this last-mentioned embodiment, said hair treatment agents include, with respect to the weight of the agent, 0.0001 to 0.01 wt. % of oligopeptide A and 0.0001 to 0.001 wt. % of oligopeptide B.

The oligopeptides used in the context of the present invention, which satisfy the aforementioned conditions, can advantageously be obtained from keratinous materials. According to the invention, it is preferred that said oligopeptides are used in high proportions with respect to the entire keratinous peptide content of the agents.

It is exceedingly preferred that the highest possible proportion of all keratinous peptides included in the agent according to the invention satisfies the aforementioned conditions.

Preferred hair treatment agents according to the invention are characterized in that at least 0.1 wt. %, preferably at least 0.5 wt. %, especially preferably at least 1 wt. %, more preferably at least 2.5 wt. %, even more preferably at least 5 wt. %, and particularly at least 10 wt. % of all keratinous peptides included in the agent have the amino acid sequence Glu-Glu-Glu.

More highly preferred hair treatment agents according to the invention are characterized in that at least 0.1 wt. %, preferably at least 0.5 wt. %, especially preferably at least 1 wt. %, more preferably at least 2.5 wt. %, even more preferably at least 5 wt. %, and particularly at least 10 wt. % of all keratinous peptides included in the agent have the amino acid sequence SEQ ID NO:2 Glu-Glu-Glu-Ile.

Even more highly preferred hair treatment agents according to the invention are characterized in that at least 0.1 wt. %, preferably at least 0.5 wt. %, especially preferably at least 1 wt. %, more preferably at least 2.5 wt. %, even more preferably at least 5 wt. %, and particularly at least 10 wt. % of all keratinous peptides included in the agent have the amino acid sequence SEQ ID NO:1 Tyr-Glu-Glu-Glu.

Especially preferred hair treatment agents according to the invention are characterized in that at least 0.1 wt. %, preferably at least 0.5 wt. %, especially preferably at least 1 wt. %, more preferably at least 2.5 wt. %, even more preferably at least 5 wt. %, and particularly at least 10 wt. % of all keratinous peptides included in the agent have the amino acid sequence SEQ ID NO:3 Tyr-Glu-Glu-Glu-Ile.

Exceedingly preferred hair treatment agents according to the invention are characterized in that at least 0.1 wt. %, preferably at least 0.5 wt. %, especially preferably at least 1 wt. %, more preferably at least 2.5 wt. %, even more preferably at least 5 wt. %, and particularly at least 10 wt. % of all keratinous peptides included in the agent have the amino acid sequence SEQ ID NO:4 Tyr-Glu-Glu-Glu-Ile-Arg.

Even more highly preferred hair treatment agents according to the invention are characterized in that at least 0.1 wt. %, preferably at least 0.5 wt. %, especially preferably at least 1 wt. %, more preferably at least 2.5 wt. %, even more preferably at least 5 wt. %, and particularly at least 10 wt. % of all keratinous peptides included in the agent have the amino acid sequence SEQ ID NO:5 Tyr-Glu-Glu-Glu-Ile-Arg-Val.

Particularly preferred hair treatment agents according to the invention are characterized in that at least 0.1 wt. %, preferably at least 0.5 wt. %, especially preferably at least 1 wt. %, more preferably at least 2.5 wt. %, even more preferably at least 5 wt. %, and particularly at least 10 wt. % of all keratinous peptides included in the agent have the amino acid sequence SEQ ID NO:6 Tyr-Glu-Glu-Glu-Ile-Arg-Val-Leu.

The conditions mentioned above relate to the total content of peptides originating from keratinous materials in the agents according to the invention. Of course, other peptides and/or protein hydrolysates, for example from other natural sources, can be used in addition to the oligopeptides of keratinous origin. For example, the additional use of wheat protein hydrolysate is preferred.

The following vitamins, provitamins, and vitamin precursors and derivatives thereof should preferably be understood by suitable vitamins:

Vitamin A: Retinol (vitamin A₁) and 3,4-didehydroretinol (vitamin A2) belong to the group of substances referred to as vitamin A. β-carotene is the provitamin of retinol. For example, vitamin A acid and esters thereof, vitamin A aldehyde, and vitamin A alcohol and esters thereof such as the palmitate and the acetate are considered as a vitamin A component.

Vitamin B: The vitamin B group or the vitamin B complex includes, among other things: Vitamin B₁ (thiamine); Vitamin B₂ (riboflavin); Vitamin B₃, often used to refer to the compounds nicotinic acid and nicotinic acid amide (niacinamide); Vitamin B₅ (pantothenic acid and panthenol), in the context of this group, panthenol is preferably used, derivatives of panthenol that can be used are, in particular, the esters and ethers of panthenol, pantolactone, and cationically derivatized panthenols, individual representatives are, for example, panthenol triacetate, panthenol monoethyl ether and the monoacetate thereof, and cationic panthenol derivatives; and/or Vitamin B₆ (pyridoxine, pyridoxamine, and pyridoxal).

Vitamin C (ascorbic acid): Use in the form of the palmitic acid ester, the glucosides, or the phosphates can be preferred. Use in combination with tocopherols can likewise be preferred.

Vitamin E (tocopherols, particularly α-tocopherol).

Vitamin F: The term “vitamin F” is typically understood to mean essential fatty acids, particularly linoleic acid, linolenic acid, and arachidonic acid.

Vitamin H: The compound (3aS,4S,6aR)-2-oxohexahydrothienol[3, 4-d]imidazole-4-valeric acid is referred to as vitamin H. In the meantime, the common name biotin has become established for said compound.

Especially preferred are vitamins, provitamins, and vitamin precursors from the groups A, B, E, and H. Nicotinic acid amide, biotin, pantolactone, and/or panthenol is particularly preferred.

The weight percentage of the one or more vitamins, vitamin derivatives, and/or vitamin precursors with respect to the total weight of the hair treatment agents is preferably 0.001 to 2 wt. %, especially preferably 0.005 to 1 wt. %, and particularly 0.01 to 0.5 wt. %.

Extracts that can be produced from all parts of a plant should be understood by suitable plant extracts. These extracts are typically produced by extraction of the entire plant. However, in individual cases, it can also be preferred that the extracts are produced exclusively from flowers and/or leaves of the plant. Above all, the extracts from green tea, oak bark, nettles, witch hazel, hops, chamomile, burdock root, horsetail, whitethorn, linden blossoms, lychee, almond, aloe vera, spruce needle, Aesculus, sandalwood, juniper, coconut, mango, apricot, lime, wheat, kiwi, melon, orange, grapefruit, sage, rosemary, birch, mallow, meadowfoam, wild thyme, yarrow, garden thyme, melissa, restharrow, coltsfoot, marshmallow, ginseng, ginger root, Echinacea purpurea, Olea europea, Boerhavia diffuse roots, Foeniculum vulgaris, and Apim graveolens are suitable. Especially preferred for use in the compositions according to the invention are the extracts from green tea, nettles, witch hazel, chamomile, aloe vera, ginseng, Echinacea purpurea, Olea europea, and/or Boerhavia diffuse roots.

Water, alcohols, and mixtures thereof can be used as extracting agents for producing the mentioned plant extracts. Among the alcohols, lower alcohols such as ethanol and isopropanol, but in particular polyhydric alcohols such as ethylene glycol and propylene glycol, both as a sole extracting agent and in mixture with water, are preferred. Plant extracts based on water/propylene glycol at a ratio of 1:10 to 10:1 have proven especially suitable.

The plant extracts can be used in pure form or in diluted form. If the plant extracts are used in diluted form, the plant extracts typically include approximately 2-80 wt. % of active matter and, as a solvent, the extracting agent or extracting agent mixture used in obtaining the active matter.

The plant extracts can be used in the hair treatment agents according to the invention preferably in an amount of 0.01 to 10 wt. %, more preferably 0.05 to 7.5 wt. %, and particularly 0.1 to 5 wt. % (with respect to the total weight of the agents).

Glycerol may be added to the hair cleaning and care agents separately in an amount of up to 10 wt. % (with respect to the total weight of the agent). However, glycerol can also be a constituent of the previously mentioned aqueous-alcoholic carrier.

It has been determined that the hair treatment agents according to the invention are also suitable for use as an anti-dandruff preparation.

The total weight of anti-dandruff agents with respect to the total weight of the hair treatment agents can be preferably 0.01 to 10 wt. %, more preferably 0.025 to 7.5 wt. %, especially preferably 0.05 to 5 wt. %, and particularly 0.075 to 3 wt. %.

Suitable anti-dandruff active substances can be selected from piroctone olamine, climbazole, zinc pyrithione, ketoconazole, salicylic acid, sulfur, selenium sulfide, tar preparations, undecenoic acid derivatives, burdock root extracts, Populus extracts, nettle extracts, walnut shell extracts, birch extracts, willow bark extracts, rosemary extracts, and/or arnica extracts. Climbazole, zinc pyrithione, and piroctone olamine are preferred.

Further active substances, auxiliary substances, and additives that may preferably be included in the hair treatment agents according to the invention are, for example: humectants; perfumes; UV filters; thickeners such as gelatin or plant gum, for example agar-agar, guar gum, alginates, xanthan gum, gum arabic, gum karaya, locust bean gum, linseed gums, dextrans, cellulose derivatives, e.g., methyl cellulose, hydroxyalkyl cellulose, and carboxymethyl cellulose, starch fractions and derivatives such as amylose, amylopectin, and dextrins, clays and phyllosilicates such as bentonite, or fully synthetic hydrocolloids such as polyvinyl alcohol, the Ca, Mg, or Zn soaps; structurants such as maleic acid and lactic acid; dimethyl isosorbide; cyclodextrins; fiber-structure-improving active substances, particularly mono-, di-, and oligosaccharides, such as glucose, galactose, fructose, fruit sugar, and lactose; dyes for dyeing the agent; active substances such as bisabolol and/or allantoin; complexing agents such as EDTA, NTA, β-alanine diacetic acid, and phosphonic acids; ceramides, understood to mean N-acylsphingosine (fatty acid amides of sphingosine) or synthetic analogs of such lipids (pseudo-ceramides); propellants such as propane-butane mixtures, N₂O, dimethyl ether, CO₂, and air; antioxidants; and/or additional viscosity regulators such as salts (NaCl).

The agents according to the invention are preferably rinse-off products, i.e., said agents are rinsed out of the hair after a certain exposure time. Said exposure time is preferably less than one hour, i.e., the consumer preferably does not leave the products in the hair until the next hair wash.

Therefore, the present invention also relates to a method for treating hair, in which method an agent according to the invention is applied to dry or moist hair, left there for a time period of 30 to 300 seconds, and then rinsed out.

The agents according to the invention lead to considerably greater strengthening of the internal and external hair structure. Therefore, the present invention also relates to the use of agents according to the invention to strengthen the hair structure, particularly the inner hair structure.

In the sense of the invention, the term “structure strengthening” is understood to mean a reduction in the damage to keratin fibers caused by a wide range of influences. The restoration of the natural strength, for example, plays an important role here. Restructured fibers are distinguished, for example, by improved shine, improved texture, and improved combability. In addition, restructured fibers have optimized strength and elasticity. Successful structure strengthening or restructuring can be demonstrated physically as a melting-point increase in comparison with the damaged fiber.

It is also possible that the agents according to the invention are formulated as an active-substance combination of the ingredients a) to d) and include no further ingredients except a cosmetic carrier. Such active-substance combinations can then be added to other hair treatment agents, and such active-substance combinations increase the conditioning performance of said other hair treatment agents considerably and in a synergistic manner.

Therefore, the present invention also relates to the use of mixtures of: at least one fatty alcohol, at least one cationic surfactant, at least one silicone, and 10-undecenoic acid (undecylenic acid) and/or salts of undecylenic acid to increase the hair-conditioning effect of hair treatment agents.

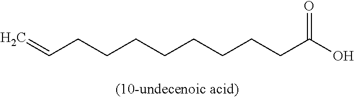

The statements made with regard to the agents according to the invention apply, mutatis mutandis, with regard to preferred embodiments of the method according to the invention and of the uses according to the invention.

### EXAMPLES

All specifications in wt. % of the composition (agent).

### Hair Treatments

| | 1-1 | 1-2 | 1-3 | 1-4 | 1-5 | 1-6 |
|---|---|---|---|---|---|---|
| Cetearyl alcohol | 7.0 | 7.0 | 7.0 | 7.0 | 7.0 | 7.0 |
| Quaternium-87 | 0.9 | 0.9 | 0.9 | 0.9 | 0.9 | 0.9 |
| Cetrimonium chloride | 1.5 | 1.5 | 1.5 | 1.5 | 1.5 | 1.5 |
| Distearoylethyl Hydroxyethyl- | 2.0 | 2.0 | 2.0 | 2.0 | 2.0 | 2.0 |
| monium Methosulfate | | | | | | |
| Polyquaternium-10 | 0.1 | 0.1 | 0.1 | 0.1 | 0.1 | 0.1 |
| Baobab seed oil | 0.1 | 0.1 | 0.1 | 0.1 | 0.1 | 0.1 |
| Polyquaternium-37 | 0.4 | 0.4 | 0.4 | 0.4 | 0.4 | 0.4 |
| Hydrolyzed Keratin | 0.5 | 0.5 | 0.5 | 0.5 | 0.5 | 0.5 |
| Glycol Distearate | 1.0 | 1.0 | 1.0 | 1.0 | 1.0 | 1.0 |
| Citric acid | 1.5 | 1.5 | 1.5 | 1.5 | 1.5 | 1.5 |
| Dimethicone | 1.0 | 1.0 | 1.0 | 1.0 | 1.0 | 1.0 |
| Undecylenic acid | 0.1 | 0.1 | 0.1 | 0.1 | 0.1 | 0.1 |
| o-Phenylphenol | — | 0.2 | 0.2 | 0.2 | 0.2 | 0.2 |
| o-Cymen-5-ol | — | 0.06 | 0.06 | 0.06 | 0.06 | 0.06 |
| Ethyl lauroyl arginate | — | 0.3 | 0.3 | 0.3 | 0.3 | 0.3 |
| Dimethyloxazolidine | — | 0.05 | 0.06 | 0.07 | 0.08 | 0.1 |
| Ethyl lauroyl arginate | — | 0.4 | 0.3 | 0.2 | 0.1 | 0.05 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 0.05 | 0.1 | 0.25 | 0.05 | 0.05 |
| Propionic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | Add to 100 |

### Hair Conditioning Agents

| | 2-1 | 2-2 | 2-3 | 2-4 | 2-5 | 2-6 |
|---|---|---|---|---|---|---|
| Cetearyl alcohol | 5.0 | 5.0 | 5.0 | 5.0 | 5.0 | 5.0 |
| Quaternium-87 | 0.75 | 0.75 | 0.75 | 0.75 | 0.75 | 0.75 |
| Behentrimonium chloride | 1.0 | 1.0 | 1.0 | 1.0 | 1.0 | 1.0 |
| Behenoyl PG-trimonium | 1.5 | 1.5 | 1.5 | 1.5 | 1.5 | 1.5 |
| chloride | | | | | | |
| Glycol Distearate | 1.0 | 1.0 | 1.0 | 1.0 | 1.0 | 1.0 |
| Distearoylethyl Hydroxyethyl- | 0.3 | 0.3 | 0.3 | 0.3 | 0.3 | 0.3 |
| monium Methosulfate | | | | | | |
| Amodimethicone/Morpholino- | 0.1 | 0.1 | 0.1 | 0.1 | 0.1 | 0.1 |
| methyl Silsesquioxane | | | | | | |
| Copolymer | | | | | | |
| Citric acid | 0.5 | 0.5 | 0.5 | 0.5 | 0.5 | 0.5 |
| Shea Butter | 1.5 | 1.5 | 1.5 | 1.5 | 1.5 | 1.5 |
| Lactic acid | 0.1 | 0.1 | 0.1 | 0.1 | 0.1 | 0.1 |
| Magnesium chloride | 0.5 | 0.5 | 0.5 | 0.5 | 0.5 | 0.5 |
| Sodium undecylenate | 0.2 | 0.2 | 0.2 | 0.2 | 0.2 | 0.2 |
| Phenoxyethanol | — | 0.5 | 0.5 | 0.5 | 0.5 | 0.5 |
| Dimethyloxazolidine | — | 0.05 | 0.06 | 0.07 | 0.08 | 0.1 |
| Glutaraldehyde | — | 0.05 | 0.06 | 0.07 | 0.08 | 0.1 |
| Hexetidine | — | 0.05 | 0.06 | 0.07 | 0.08 | 0.1 |
| Ethyl lauroyl arginate | — | 0.05 | 0.05 | 0.05 | 0.05 | 0.05 |
| Sodium o-phenylphenolate | — | 1.0 | 0.5 | 0.25 | 0.2 | 0.1 |
| o-Cymen-5-ol | — | 0.2 | 0.1 | 0.2 | 0.1 | 0.1 |
| Phenoxyisopropanol | — | 0.05 | 0.1 | 0.25 | 0.05 | 0.05 |
| Propionic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | Add to 100 |

1. A hair treatment agent, comprising:
at least one fatty alcohol;
at least one cationic surfactant, wherein the cationic surfactant comprises an alkyltrimethyl ammonium halide chloride with antimicrobial activity:
at least one silicone, wherein the at least one silicone comprises 0.01% to 20% by weight of the agent of silicones) of formula (I), 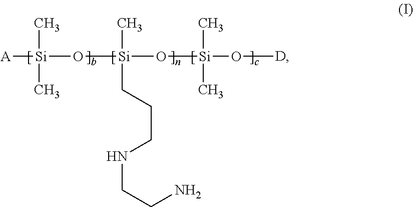
in which
A represents a group selected from: —OH, —O—Si(CH₃)₃, —O—Si(CH₃)₂OH, and —O—Si(CH₃)₂OCH₃:
D represents a group selected from: —H, —Si(CH₃)₃, —Si(CH₃)₂OH, —Si(CH₃)₂OCH₃;
b, n, and c are integers between 0 and 1000, such that
n>0,
b+c>0, and
A=—OH and/or D=−H; and
10-undecenoic acid and/or a salt thereof.

2. The agent of claim 1, wherein the at east one fatty alcohol comprises 0.1% to 20% by weight of the agent.

3. The agent of claim 1, wherein the at least one fatty alcohol comprises 2% to 8% by weight of the agent.

4. The agent of claim 1, wherein the at least one fatty alcohol comprises 2% to 8% be weight of the agent of fatty alcohol(s) of formula (II),
H₃C—(CH₂)ₖ—CH₂—OH (II),
in which k is an integer from 4 to 28.

5. The agent of claim 1, wherein at least one cationic surfactant comprises 0.05% to 20% by weight of the agent.

6. The agent of claim 1, wherein the at least one cationic surfactant comprises 0.5% to 7% by weight of the agent.

7. The agent of claim 1, wherein the at least one cationic surfactant comprises 0.5% to 7% by weight of the agent of behenyltrimethylammonium chloride.

8. (canceled)

9. The agent of claim 1, wherein the at least one silicone comprises 0.1% to 5% by weight of the agent.

10. (canceled)

11. The agent of claim 1, wherein 10-undecenoic acid comprises 0.001% to 1% by weight of the agent.

12. The agent of claim 1, wherein 10-undecenoic acid comprises 0.1% to 0.2% by weight of the agent.

13. The agent of claim 1, wherein 0.001% to 1% by weight of the agent is salt selected from a group consisting of: ammonium undecylenate, sodium undecylenate, potassium undecylenate, magnesium undecylenate, calcium undecylenate, zinc undecylenate, manganese undecylenate, and mixtures thereof.

14. The agent of claim 1, wherein 0.1% to 0.2% by weight of the agent is salt selected from a group consisting of: ammonium undecylenate, sodium undecylenate, potassium undecylenate, magnesium undecylenate, calcium undecylenate, zinc undecylenate, manganese undecylenate, and mixtures thereof.

15. A hair treatment agent, comprising:
2% to 8% by weight of the agent of at least one fatty alcohol;
0.5% to 7% by weight of the agent of at least one cationic surfactant, wherein the at least one cationic surfactant comprises an alkyltrimethyl ammonium chloride with antimicrobial activity:
0.1% to 5% by weight of the agent of at least one silicone, wherein the at least one silicone comprises a silicone of formula (I), 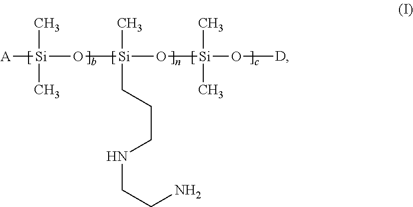
in which
A represents a group selected from: —OH, —O—Si(CH₃)₃, —O—Si(CH₃)₂OH, and —O—Si(CH₃)₂OCH₃:
D represents a group selected from: —H, —Si(CH₃)₃, —Si(CH₃)₂OH, —Si(CH₃)₂OCH₃;
b, n, and c are integers between 0 and 1000, such that
n>0,
b+c>0, and
A=—OH and/or D=−H; and
0.1% to 0.2% by weight of the agent of 10-undecenoic acid and/or a salt thereof.

16. The agent of claim 14, wherein the 10-undecenoic acid and/or salts thereof comprise a salt selected from a group consisting of: ammonium undecylenate, sodium undecylenate, potassium undecylenate, magnesium undecylenate, calcium undecylenate, zinc undecylenate, manganese undecylenate, and mixtures thereof.

17. (canceled)

18. The agent of claim 14, wherein the at least one cationic surfactant comprises behenyltrimethylammonium chloride.

19. The agent of claim 14, wherein the at least one fatty alcohol comprises an alcohol of formula (II),
H₃C—(CH₂)ₖ—CH₂—OH (II),
which k is an integer from 4 to 28.

20. A method of treating hair, comprising:
applying a hair treatment agent of claim 1 to hair; and
rinsing the agent from the hair after 30 to 300 seconds contact with the hair.

21. (canceled)


SEQUENCE LISTING (10 sequences)

SEQ ID NO: 1 - protein, 4 aa, Triticum aestivum
YEEE

SEQ ID NO: 2 - protein, 4 aa, Triticum aestivum
EEEI

SEQ ID NO: 3 - protein, 5 aa, Triticum aestivum
YEEEI

SEQ ID NO: 4 - protein, 6 aa, Triticum aestivum
YEEEIR

SEQ ID NO: 5 - protein, 7 aa, Triticum aestivum
YEEEIRV

SEQ ID NO: 6 - protein, 8 aa, Triticum aestivum
YEEEIRVL

SEQ ID NO: 7 - protein, 9 aa, Triticum aestivum
LYEEEIRVL

SEQ ID NO: 8 - protein, 5 aa, Triticum aestivum
EEEIR

SEQ ID NO: 9 - protein, 6 aa, Triticum aestivum
EEEIRV

SEQ ID NO: 10 - protein, 7 aa, Triticum aestivum
EEEIRVL